(19) United States
(12) Patent Application Publication
(10) Pub. No.: US 2005/0020665 A1
(43) Pub. Date: Jan. 27, 2005

(54) Pharmaceutical composition for treating multiple sclerosis

(76) Inventor: Ingo Neu, Rohrau (DE)

Correspondence Address: POTTER ANDERSON & CORROON LLP;ATTN: KATHLEEN W. GEIGER, ESQ., P.O. BOX 951, WILMINGTON, DE, 19899-0951, US

(21) Appl. No.: 10/470,212

(22) PCT Filed: Jan. 23, 2002
(86) PCT No.: PCT/EP02/00653

(30) Foreign Application Priority Data
Jan. 26, 2001 (DE) 101 03 506.3

(51) Int. Cl. A61K 31/405; A61K 31/381; A61K 31/343

(52) U.S. Cl. 514/419; 514/469; 514/443

(57) ABSTRACT

Use of a compound of the general formula I and/or a pharmaceutically acceptable salt thereof as active ingredient for the preparation of a pharmaceutical composition for the treatment of multiple sclerosis in humans: 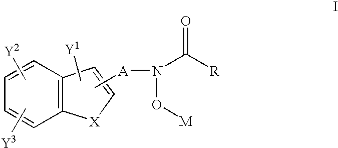 wherein
X represents NCH₃, O or S.
A represents an unsubstituted C₁₋₄ alkylene or a substituted C₁₋₄ alkylene that is mono- or poly-substituted with substituents independently selected from CH₃, CH₂CH₃, CH₂CH₂CH₃ und CH(CH₃)₂,
M represents H; benzoyl; unsubstituted C₁₋₁₂ alkanoyl; substituted C₁₋₁₂ alkanoyl that is mono- or poly-substituted with substituents independently selected from CH₃, CH₂CH₃, F and Cl; unsubstituted C₁₋₁₂ alkyl; or substituted C₁₋₁₂ alkyl that is mono- or poly-substituted with substituents independently selected from CH₃, CH₂CH₃, F and Cl;
R represents CH₃, CH₂CH₃ or NR¹R², wherein R¹ and R² independently represent H, CH₃ or CH₂CH₃, and
Y¹, Y² and Y³ are independently selected from H, CH₃, CH₂CH₃, F and Cl.

The present invention relates to the use of organic compounds for the preparation of a pharmaceutical composition for the treatment of multiple sclerosis in humans.

### BACKGROUND OF THE INVENTION

Multiple sclerosis (encephalomyelitis disseminata) (MS) is one of the most frequent nervous diseases. Its cause is not yet known. Up to the present day, there is no causal therapy at hand, but many attempts have been made to alleviate the effects of the disease. The treatment, limited in time, of the aggravation of MS by attacks with cortisone preparations has been found thereby to be helpful.

In spite of intense treatment attempts for relapse prophylaxis of MS using immuno-suppressing medicaments, such as Imurek®, and for the immuno-modulation with β-interferone preparations, such as Betaferon®, no convincing breakthrough has yet been obtained to hold up the progression of MS.

In northern Europe, Canada and the northern states of the United States, MS is the most frequent progressing disease of the central nervous system. The age when the illness strikes is between 20 and 40 years, women are to a slight extent more often affected than men.

The chronic inflammatory reaction of MS is characterized by complex interactions between cellular and humoral elements of the immune system, adhesion molecules on endothelial cells and imflammatory cells as well as a series of low-molecular mediators of inflammation. Among the mediators of inflammation, cytokins play animportant role.

The symptoms of MS are variegated and comprise in the prodromal stage inter alia pseudo-neurasthenic symptoms, cerebral nerve failures (retrobulbar neuritis, ophthalmoplegia, syllabism), spasticity, cerebellum symptoms, paraesthesia, bladder and rectum dysfunction; euphoria, the development of a subsequent dementia, is possible.

Despite intense research, the aetiology of the chronic inflammatory development has remained unclear. Essential pathogenetic mechanisms could be elucidated in the last two decades by pathologic-anatomic and immunologic studies in patients as well as by experimental work with an animal model of the MS, the experimental allergic encephalomyelitis (EAE). It is assumed that auto-immune reactions to myelin antigens mediated by T-cells play an important role.

Since the cause of MS still remains unknown up to the present day, the treatment of this disease has always proven to be problematic. The controversial situation of MS therapy (Bauer, H. I.: “Nervenarzt”, 54, 1983, 400 to 405) has been revealed in more than 100 attempts at treatment which have been carried out, at least at times, in medicine, including unconventional methods and methods by outsiders.

Some authors have postulated an infectious cause of MS and one hypothesis assumes that viruses could play a significant role. When a viral infection was assumed, the administered therapeutic agents, in particular amantadin or methisazon, did not show convincing results. The use of interferon has also been discussed in many cases, however, the results of the treatment were not convincing as regards the progress of the disease.

Other principles of treatment are based on metabolic theories. The assumptions of possible protein metabolism disturbances are based on the presence of a number of biogenetic amines which could not be found in healthy human beings.

The metabolism hypothesis is based on trials with highly unsaturated fatty acids (omega fatty acids), since a lower MS prevalence is observed when there is a higher consumption of vegetable oil or fish.

Other studies are based on thrombocyte aggregation, which is significantly increased in MS patients during the attacks of the disease. This has led to treatment tests with aggregation-inhibiting therapeutic substances.

Finally, the attempt was made to use cortico-steroids and immune suppressors in therapeutic methods which could attenuate in the acute early phase the intensity and duration of the corresponding phases of the attacks. Due to the great adverse effects of the cortico-steroids, ACTH, etc., an application is only possible which is limited in time, although this application cannot prevent further attacks. At present, the pulse therapy with cortisone preparations during the attack has become accepted.

Apart from the above-mentioned therapies, a number of unconventional methods has been tried, including acupuncture, special diets, mega-vitamin therapies, etc. In addition, 15 to 20 years ago the oxygen overpressure therapy was propagated (B. Fischer, New England J. Med., 303, 1983, 181 to 186).

The difficulties of MS research and therapy are, on the one hand, due to the fact that the causes and the elicitor of the disease are unknown. There is an animal model for testing medicaments, the experimental allergic encephalomyelitis (EAE), however, the results thereof cannot automatically be transferred to humans.

In Acta Neurol. Scand. 75, 1987, 361-363 (Neu, et al.), the release of leucotrienes from neutrophils and from suspensions of thrombocytes and neutrophils in reaction to Ionophor A23187 was measured. Based on the results of this scientific study, it was postulated that the release of the sulfidopeptid leucotriene could be involved in the perivenous plaque formation in MS. It is further stated therein that the suppression of the leucotriene. synthesis by medicaments might be a promising therapeutic concept if these results could be confirmed for a greater number of MS patients and in comparisons with other neurological diseases in EAE trials. This literature source allows the conclusion to be drawn that the scientific observations have not been made with a representative number of patients and that therefore conclusions are not possible. Although comparisons with other neurological diseases have been proposed, the exact genesis of these diseases was not given. Further, tests on EAE animals were considered necessary, although it is known that the results of these test systems can only be transferred to MS patients to a limited extert. This was, in the end, again confirmed e.g. for the antimetabolite azothioprin (Imurek®), which showed promising indications in the E animal test, but was not convincing in the treatment of MS patients.

Further, the attempt was made to exert influence on the progression of MS via a pharmacological blockade with leucotriene biosynthesis inhibitors and leucotriene receptor antagonists. In the animal model it was possible to suppress the inflammatory reactions of MS via a pharmacological blockade of leucotriene C₄ with chemical leucotriene inhibitors and sulphosalacines. These sulphosalazines (Azulfidine®), which were successful in the animal model, were tested on MS patients in a clinical study. In the first 18 months of the treatment with azulfidine in a double blind study carried out in the United States and Canada, an exceptionally beneficial effect could be proven. However, three years after the end of the study this effect could no longer be proven. Here, the problem of the limited transferability of the results of test systems on animals to MS patients was again to be observed.

A further study also tried to use a leucotriene inhibiting substance. In this study, the Boswellia acid (Boswellia serrata) was tested, a component of the incense resin, which is predominantly used for treating rheumatoid polyarthritis, bronchial asthma and colitis ulcerosa. A good leucotriene inhibiting effect was observed in vitro. No success could, however, be obtained with the animal experimental study of the Boswellia acid. A treatment attempt with MS patents was therefore superfluous.

In summary, it has been shown that no conclusive method is to be found in the literature, in, which specific active ingredients could be used in a successful therapy of MS.

### OBJECT OF THE INVENTION

It is therefore an object of the invention to provide an active ingredient for the preparation of a pharmaceutical composition for the treatment of MS in humans.

### INVENTION

The object of the present invention is solved by the use of a compound of the general formula I and/or a pharmaceutically acceptable salt thereof as active ingredient for the preparation of a pharmaceutical composition for the treatment of multiple sclerosis in humars: 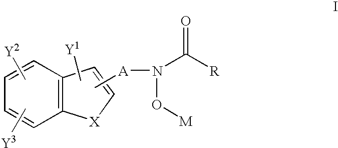
wherein
X represents KCH₃, O or S,
A represents an unsubstituted C₁₋₄ alkylene or a substituted C₁₋₄ alkylene that is mono- or poly-substituted with substituents independently selected from CH₃, CH₂CH₃, CH₂CH₂CH₃ und CH(CH₃)₂,
M represents H; benzoyl; unsubstituted C₁₋₁₂ alkanoyl; substituted C₁₋₁₂ alkanoyl that is mono- or poly-substituted with substituents independently selected from CH₃, CH₂CH₃, F and Cl; unsubstituted C₁₋₁₂ alkyl; or substituted C₁₋₁₂ alkyl that is mono- or poly-substituted with substituents independently selected from CH₃, CH₂CH₃, F and Cl;
R represents CH₃, CH₂CH₃ or NR¹R², wherein R¹ and R² independently represent H, CH₃ or CH₂CH₃, and
Y¹, Y² and Y³ are independently selected from H, CH₃, CH₂CH₃, F and Cl.

It was surprisingly found that the frequency of the attacks and the chronic progression decreased when a compound of the general formula I and/or a pharmaceutically acceptable salt and/or a hydrate thereof was administered to patients ill with multiple sclerosis. This result is unexpected since the causes, the symptoms and the behaviour of MS and of the persons ill therewith are either unknown or very complex.

The compounds of formula I according to the invention preferably used include those compounds wherein, independently or in one or several combinations,
wherein
M represents H,
at least two of Y¹, Y² and Y³ represent H,
A represents CH₂ or CHCH₃, and
X represents S.

The active ingredient used according to the invention can be used as such and/or in the form of pharmaceutically acceptable salts thereof.

If sterical isomers, such as optical isomers or conformation isomers, or hydrates and complexes of the compounds of the invention exist, the present invention includes each and all of them. The compounds of the present invention and the methods for preparing the same are disclosed e.g. in U.S. Pat. No. 4 873 259.

Examples of the compounds of the general formula I used in accordance with the present invention are
N-hydroxy-N-(1-benzo[b]thien-2-ylethyl) acetamide,
1-(1-Benzo[b]thien-2-ylethyl)-1-hydroxy urea,
N-hydroxy-N-(1-benzo[b]thien-2-ylethyl)-N′-methyl urea,
N-hydroxy-N-(1-benzo[b]thien-2-ylethyl)-N′,N′-dimethyl urea,
N-hydroxy-N-benzo[b]thien-2-yl;methyl urea,
N-hydroxy-N-benzo[b]thien-2-ylmethyl-N′-methyl urea,
N-hydroxy-N-benzo[b]thien-2-ylmethyl-N′,N′-dimethyl urea,
N-hydroxy-N-(1-benzo[b]thien-3-ylethyl) acetamide,
N-hydroxy-N-(1-benzo[b]thien-3-ylethyl) urea,
N-hydroxy-N-[1-(3-methylbenzo[b]thien-2-yl)-ethyl]urea,
N-hydroxy-N-(1-benzo[b]fur-2-ylethyl) acetamide,
N-hydroxy-N-(1-benzo[b]fur-2-ylethyl) urea,
N-hydroxy-N-[1-(1-methylindol-3-yl)-ethyl]acetamide,
N-hydroxy-N-[1-(1-methylindol-3-yl)-ethyl]urea,
N-hydroxy-N-[1-(1-methylindol-3-yl)-ethyl]-N′-methyl urea,
N-hydroxy-N-[1-(5-chlorbenzo[b]fur-2-yl)-ethyl]urea,
N-hydroxy-N-[1-(5-fluorbenzo[b]thien-2-yl)-ethyl]urea.

The use according to the invention of:
N-hydroxy-N-(1-benzo[b]thien-2-ylethyl) acetamide,
1-(1-benzo[b]thien-2-ylethyl)-1-hydroxy urea,
N-hydroxy-N-(1-benzo[b]thien-2-ylethyl)-N′-methyl urea, or
N-hydroxy-N-(1-benzo[b]thien-2-ylethyl)-N′,N′-dimethyl urea is preferred.

Particularly preferred is the use according to the invention of 1-(1-benzo[b]thien-2-ylethyl)-1-hydroxy urea, in the following also designated zileuton:

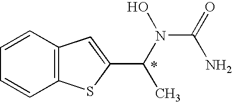

Zileuton can either be used as a racemic mixture of the R(+)- and S(−) enantiomers or in the form of its pure enantiomers. The use of the racemate is preferred.

Usually, the active ingredient is used in an amount of 0.001 to 2.0 mg, preferably in an amount of 0.01 to 2.0 mg, more preferably in an amount of 0.1 to 1.0 mg, and most preferably in an amount of 600 mg per dose. The administration is performed once to five times daily, preferably four times daily.

Therapeutic Forms of Administration

The preparation of pharmaceutical compositions having a content of a compound of the general formula I and/or a pharmaceutically acceptable salt thereof or their use in the utilization according to the invention is carried out in the common manner by means of conventional pharmaceutic-technological methods. Advantageous is e.g. the processing together with suitable, pharmaceutically acceptable adjuvants and/or carrier substances in the pharmaceutical forms which are suitable for the various indications and types of application. It could also be an advantage if the medicament comprises further active ingredients.

One important systemic form of application is the peroral administration in the form of tablets, hard or soft gelatine capsules, dragees, powder, pellets, micro-capsules, oblong compressed tablets, granulates, chewable tablets, sucking tablets, chewing gum, sachets or globuli.

As adjuvants for the preparation of pharmaceutical compositions for peroral admiristration, the following are, for example, used: double-side adhesives and lubricants and separating agents, dispersion agents, such as e.g. flame-dispersing silicon dioxide, disintegrants, such as e.g. various kinds of starch, PVP, cellulose ester as a granulating agent, such as e.g. wax-like and/or polymeric materials used on the basis of Eudragit®, cellulose or Cremophor®.

Antioxidants, sweetening agents, such as e.g. saccharose, xylite oder mannitol, taste correctors, flavouring agents, preservatives, colouring agents, buffer substances, topical application agents, such as e..g. microcrystalline cellulose, starch and starch hydrolisates (e.g. Celutab®), lactose, polyethylene glycoles, polyvinyl pyrrolidone and di-calcium phosphate, lubricants, fillers, such as e.g. lactose or starch, binders in the form of lactose, types of starch, such as wheat or corn or rice starch, cellulose derivatives, such as e.g. methyl cellulose, hydroxypropyl cellulose or silica, talcum, stearates, such as e.g. magnesium stearate, aluminium stearate, calcium stearate, talc, siliconized talc, stearine acid, cetyl alcohol, hydrated fats, may also be used.

The common emulsions, gels, ointments, creams or mixed-phase or amphipathic emulsion systems (oil/water-water/oil mixed phase) as well as liposomes and transferosomes can also be mentioned for a conventional application to the skin.

For example, as adjuvants or carriers sodium alginate is suited as a gelatinizing agent for the preparation of a suitable base, or cellulose derivatives, such as e.g. guar or xanthane gum, an organic gelatinizing agents, such as e.g. aluminium hydroxides or bentorites (so-called thixotropic gelatinizing agents), polyacrylic acid derivatives, such as e.g. Carbopol®, polyvinyl pyrrolidone, microcrystalline cellulose or carboxymethyl cellulose. Further, amphipatic low- and high-molecular compounds such as phospholipids are to be considered. The gels can be present either as hydrogels on a water basis or as hydrophobic organogels, e.g. on the basis of mixtures of low- and high-molecular paraffin hydrocarbons and vaseline.

Anionic, cationic or neutral tensides, e.g. alkali soaps, metal soaps, amine soaps, sulfurated and sulfonated compounds, invert soaps, higher fatty alcohols, partial fatty acid esters of sorbitan and polyoxyethylene sorbitan, wool wax, lanolin or other synthetic products may be used as emulgators for the preparation of the oil/water and/or water/oil emulsions.

The hydrophilic organogels can be prepared e.g. on the basis of high-molecular polyethylene glycols. These gel-like forms can be washed off. Vaseline, natural or synthetic waxes, fatty acids, fatty alcohols, fatty acid esters, e.g. as mono-, di- or triglycerides, paraffin oil or vegetable oils, hardened castor oil or coconut oil, lard, synthetic fats, e.g. on the basis of capryl, capron, laurate and stearic acid, or triglyceride mixtures such as Miglycol®, are used as lipids in the form of fat-like and/or oil-like and/or wax-like components for the preparation of ointments, creams or emulsions.

Osmotically effective acids and bases, e.g. hydrochloric acid, citric acid, sodium hydroxide solution, caustic potash solution, sodium hydrogen carbonate, further buffer systems, such as e.g. citrate, phosphate, tris-buffer or triethanolamine may be used for the adjustment of the pH value.

Preservatives, e.g. methyl or propylbenzoate (parabene) or sorbic acid may also be added in order to increase the stability.

Pastes, powders or solutions can be mentioned as further topically applicable forms. As the consistency-forming base, the pastes frequently contain lyophilic and hydrophilic adjuvants having a very high proportion of solids. In order to increase the dispersing effect, the flowability and lubricating property and to avoid forming agglomerates, the powders or topically applicable powders may contain e.g. types of starch, such as wheat starch or rice starch, flame dispersing silicon dioxide or silica, which also serve as diluents.

In particular, plasters may be produced as transdermal systems which are able to release the active ingredient in a controlled manner over a longer or shorter period of time on the basis of various layers and/or mixtures of suitable adjuvants and carriers. For the purpose of an improved and/or accelerated penetration, those substances are used in the preparation of such transdermal systems, which enhance the membrane permeation, or permeation promoters, such as e.g. oil acid, Arzone®, adipin acid derivatives, ethanol, urea, propylglycol, together with suitable adjuvants and carriers, such as solvents, polymer components, e.g. on the basis of Eutragit®.

Further, injections may also be administered. They are prepared either in the form of ampoules or also as so-called injections ready for use, e.g. as prepared syringes or disposable syringes and, in addition to this, for a repeated extraction, also in septum flasks. The injections may be administered in the form of a subcutaneous (s.c.), intramuscular (i.m.), intravenous (i.v.) or intracutaneous (i.c.) application. The respective appropriate forms of injections may be prepared in particular as solutions, crystal suspensions, nano-particular or colloid-dispersing systems, such as e.g. hydrosols.

The preparations to be injected may also be prepared as concentrates which may be adjusted with aqueous isotonic diluents to the desired active ingredient dosage. They may further be produced as powders, such as e.g. lyophylisates, which are then, preferably prior to application, dissolved with suitable diluents or are dispersed.

As adjuvants and carriers when preparing injectable preparations aqua sterilisata may be used as well as substances which have an influence on the pH value, e.g. organic and inorganic acids and bases as well as salts thereof, buffer substances for adjusting the pH value, isotonisation agents, such as e.g. sodium chloride, sodium hydrogen carbonate, glucose and fructose, tensides or surfactants and emulgators, such as e.g. partial fatty acid esters of polyoxyethylene sorbitans (Tween®), or e.g. fatty acid esters of polyoxyethylene (Cremophor®), fatty oils, such as e.g. peanut oil, soybean oil and castor oil, synthetic fatty acid esters, such as e.g. ethyloleate, isopropyl myristate and neutral oil (Miglycol®), as well as polymeric adjuvants, such as e.g. gelatine, dextran, polyvinyl pyrrolidone, additives of organic solvents increasing solvability, such as e.g. propylene glycol, ethanol, N,N-dimethyl acetamide, propylene glycol or completing agents, such as e.g. citrates and urea, preservatives, such as e.g. benzene acid hydroxypropyl and methyl ester, benzene alcohol, antioxidants, such as e.g. sodium sulfite and stabilizers, such as e.g. EDTA.

The skilled person may easily recognize and prepare the respective suitable forms of the medicament in accordance with the rules and regulations for prescriptions and the procedures on the basis of pharmaceutical-physical principles.

The use of zileuton in the form von Zyflo™Filmtab® tablets has proven to be particularly advantageous. This substance is indicated in adults and children of 12 and older for the prophylaxis of asthma and the treatment of chronic asthma and is authorized in the United States for this indication. The use of zileuton for the preparation of Zyflo™Filmtab® tablets, as are distributed by the Abbott laboratories in the United States for the treatment of multiple sclerosis in humans, is also included in the present invention.

Experimental Part

15 patients having clinically active MS were treated with zileuton in a treatment trial. 15 MS patients without this treatment served as a control. One tablet of Zyflo™Filmtab® was administered to each four times a day. The study had a duration of three years. As compared with the 15 MS patients without this treatment, the frequency of attacks and the chronic progression significantly decreased in the MS patients treated with zileuton.

In order to prove the effect of the therapy, nuclear magnetic resonance spectroscopies of the brain were carried out every six months using the contrast agent Magnevist®, so that the floridity or acuteness of the centres (florides) of inflammation could be objectified. No new or acute centres absorbing the contrast agent could be determined in the MS patients treated with zileuton

1. Use of a compound of the general formula I and/or a pharmaceutically acceptable salt thereof as active ingredient for the preparation of a pharmaceutical composition for the treatment of multiple sclerosis in humans: 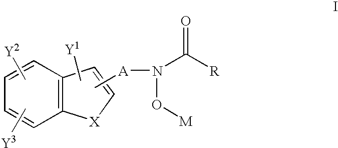
wherein
X represents NCH₃, O or S,
A represents an unsubstituted C₁₋₄ alkylene or a substituted C₁₋₄ alkylene that is mono- or poly-substituted with substituents independently selected from CH₃, CH₂CH₃, CH₂ CH₂CH₃ and CH(CH₃)₂,
M represents H; benzoyl; unsubstituted C₁₋₁₂ alkanoyl; substituted
C₁₋₁₂ alkanoyl that is mono-or poly-substituted with substitutents independently selected from alkylene that is mono- or poly-substituted with substituents independently selected from CH₃, CH₂CH₃, F and Cl; unsubstituted C₁₋₁₂ alkyl; or substituted C₁₋₁₂ alkyl that is mono- or poly-substituted with substitutents independently selected from CH₃, CH₂CH₃, F and Cl;
R represents CH₃, CH₂CH₃, or NR¹R², wherein R¹ and R² independently represent H, CH₃, or CH₂CH₃, and
Y¹, Y² and Y³ are independently selected from H, CH₃, CH₂CH₃, F and Cl.

2. Use according to claim 1, wherein M represents H.

3. Use according to claim 1, wherein at least of Y¹, Y² or Y³ represent H.

4. Use according to claim 1, wherein A represents CH₂ or CHCH₃.

5. Use according to claim 1, wherein X represents S.

6. Use according to claim 1, wherein the compound of the general formula I is selected from:
N-hydroxy-N-(l -benzo[b]thien-2-ylethyl)acetamide,
1-(1-benzo [b]thien-2-ylethyl)-1-hydroxy urea,
N-hydroxy-N-(1-benzo[b]thien-2-ylethyl)-N′-methyl urea,
N-hydroxy-N-(1-benzo[b]thien-2-ylethyl)-N′,N′-dimethyl urea,
N-hydroxy-N-l-benzo[b]thien-2-ylmethyl urea,
N-hydroxy-N-1-benzo[b]thien-2-ylmethyl-N′-methyl urea,
N-hydroxy-N-1-benzo[b]thien-2-ylmethyl-N′,N′-dimethyl urea,
N-hydroxy-N-(1-benzo [b]thien-3-ylethyl) acetamide,
N-hydroxy-N-(1-benzo [b]thien-3-ylethyl) urea,
N-hydroxy-N-[1-(3-methylbenzo[b]thien-2-yl)-ethyl]urea, and
N-hydroxy-N-[1-(5-fluorobenzo[b]thien-2-yl)-ethyl]urea.

7. Use according to claim 1, wherein the pharmaceutical composition additionally comprises one or more pharmaceutically acceptable carriers and/or one or more toxicologically safe adjuvants.

8. Use according to claim 1, wherein the pharmaceutical composition comprises further active ingredients.

9. Use according to claim 1, wherein the pharmaceutical composition is prepared in one of the following forms: in the form of an inhalation therapeutic agent, in the form of a transdermal therapeutic system, in the form of a gastro-intestinal therapeutic system, as tablet, ointment, suspension, emulsion, balm, plaster, or dosage aerosol.

10. Use according to claim 1, wherein the concentration of the active ingredient amounts to 0.001 to 2.0 mg per dose.

11. Use according to claim 10, wherein the concentration of the active ingredient amounts to 0.1 to 2.0 mg per dose.

12. Use according to claim 10, wherein the concentration of the active ingredient amounts to 0.1 to 1.0 mg per dose.

13. Use according to claim 1, wherein the medicament is prepared for one to five daily dosages.
